(19) United States
(12) Patent Application Publication
(10) Pub. No.: US 2010/0249489 A1
(43) Pub. Date: Sep. 30, 2010

(54) Intraventricular blood pumps anchored by expandable mounting devices

(76) Inventor: Robert Jarvik, New York, NY (US)

Correspondence Address: Robert Jarvik, M.D;c/o Jarvik Heart, Inc, 333 W 52nd St., New York, NY, 11019, US

(21) Appl. No.: 12/383,715

(22) Filed: Mar. 27, 2009

(51) Int. Cl. A61M 1/12

(52) U.S. Cl. 600/16

(57) ABSTRACT

An intraventricular blood pump is retained in position by an expandable stent placed in the aorta that anchors to the aortic wall. The pump ejects blood across the aortic valve either through a conduit or as a free stream of blood without a mechanical conduit passing between the valve leaflets. The ejection of blood causes a reactive force pushing the pump towards the ventricular apex and away from the valve. Thus, the pump may be held by three filaments connecting it to the anchoring stent. Other flexible members such as a tube made of pericardium, sutures, or a rigid rod may be used to hold the pump in place. The preferred embodiment includes an apically introduced stent anchored aortic valve having two flexible tissue leaflets and a conduit channeling blood from the pump in the ventricle into the aortic root and passing through the non-coronary sinus in the position usually occupied by the non-coronary cusp of the aortic valve. This device can be surgically implanted through a small incision without the need for cardiopulmonary bypass in elderly or severely ill patients who cannot safely undergo more invasive surgery. Pumps using durable bearings and hermetically sealed motors are combined with tissue valves such that the entire device is durable for many years.

### BACKGROUND OF THE INVENTION

This invention provides intraventricular blood pumps that are implanted by expandable stent fixation devices and also preserve function of the natural aortic valve or replace it with a prosthetic valve. Prior art includes miniature blood pumps implanted within the annulus of the aortic valve and sutured into position using a sewing cuff. The pump may be combined with a mechanical valve or a tissue valve as disclosed in Jarvik U.S. Pat. No. 7,479,102 entitled Minimally invasive transvalvular ventricular assist device or may be connected to a sewing ring so that two of the patient's natural leaflets may be preserved. The surgical techniques used to suture these devices in place within the aortic annulus require opening the aortic root to expose the natural valve and place the prosthesis. This requires cardiopulmonary bypass. In some patients who are too sick to under go bypass, particularly the elderly, devices are needed that can be implanted and fixed in position for long term use without cardiopulmonary bypass.

Jarvik U.S. Pat. No. 5,888,241, Cannula entitled pumps for temporary cardiac support and methods of their application and use discloses a small blood pump and cannula designed for insertion via a small incision in the apex of the ventricle, and placed so that the pump is located in the ventricle and the cannula transverses the aortic valve. This works well for short term use, but for permanent implantation has the problem that the natural valve leaflets can erode by contact with the cannula that passes between them.

Prior art discloses both blood pumps that may be affixed in position by means of expandable stents, and heart valves that can be retained in place by means of expandable stents. Barbut, et al., U.S. Pat. No. 7,144,364, entitled Endoscopic arterial pumps for treatment of cardiac insufficiency and venous pumps for right-sided cardiac support, discloses miniature blood pumps contained within expandable stents. Although these are suitable for fixing a pump in place within a large artery, they cannot be used to fix the pump within the ventricle itself, because the diameter of the ventricle is constantly increasing and decreasing as the heart beats. The present invention successfully achieves intraventricular pump placement and fixation by locating a stent in the aortic root or annulus of the aortic valve and using rigid or flexible connecting members, such as tubes, rods, or threads to hang the blood pump in the ventricle near the valve. Since the pump ejects blood into the aortic root, there is an opposing force on the pump tending to push it further towards the apex of the ventricle and away from the valve. The most minimal attachment necessary between the stent secured in the aortic root and the blood pump in the cavity of the ventricle would be a single flexible suture that would be in tension as it holds the pump. It is preferable to use three sutures to better stabilize the pump and hold it more stationary.

Siess discloses a small pump to be implanted in a blood vessel, U.S. Pat. No. 7,027,875, entitled Intravascular pump. The device includes a cannula and an expandable stent around the cannula used to enlarge the diameter of the cannula after insertion. This structure is not intended for fixation of the pump in position and would not be able to fix a blood pump within the ventricle unless it was so large it spanned the entire diameter of the aortic root. This would occlude the coronary arteries which would be fatal.

Seiss also discloses, in United States Patent Application 20090024212 entitled A method for performing intravascular cardiac surgery a method of dilating a stenosed aortic valve and implanting a stent mounted prosthetic tissue valve using a micro-axial pump positioned in the lumen of the valve during insertion. The micro-axial pump is not suited to long term use and is not affixed to the stent in order to be implanted permanently. Rather, the micro-axial pump and cannula is adapted to be removed after the tissue valve is deployed in place.

In U.S. Patent Application No. 20060074484, entitled Methods and devices for repair or replacement of heart valves or adjacent tissue without the need for full cardiopulmonary support, Huber disclosed the use of the transapical approach to implant a stent mounted aortic valve. That general approach has been used successfully in humans by others.

Additional devices and methods are known in the prior art for implantation of stent retained prostethetic valves including Iobbi, U.S. Pat. No. 7,399,315 entitled Minimally-invasive heart valve with cusp positioners, and Schreck, U.S. Pat. No. 7,381,218, entitled System and method for implanting a two-part prosthetic heart valve.

Other related prior art includes stents with vessel piercing fixation hooks or barbs that secure the device more securely than an expanded mesh alone, such as U.S. Patent Application No. 20070179591, by Baker, entitled Intra-luminal grafting system.

None of the prior art inventions sought to provide a permanently implantable blood pump that could be positioned in the cavity of the ventricle by means of less invasive surgical techniques without the need of cardiopulmonary bypass and retained by an expandable stent rather than by suturing. None of the prior art inventions recognized that a pump supported in the ventricle and configured to expel blood into the aorta would create an axial force on the retaining device in the direction opposite to the direction of blood outflow. Thus, robust stent structures having a strong attachment are necessary and the inclusion of positive fixation members such as hooks to oppose this force is functionally important. In one embodiment of the invention, the pump is “hung” within the ventricle with its outflow opening closely adjacent to the valve leaflets but without touching them and without using any graft or cannula to cross the aortic valve. In this configuration the force of the blood stream exiting the pump holds open one or more leaflets during diastole to permit blood to be expelled throughout the cardiac cycle. During systole, the valve leaflets all open due to expulsion of blood by ventricular contraction.

In the preferred embodiment of the present invention, the pump is “hung” within the left ventricle by its outflow graft, which is made of pericardium, the same material being used for the prosthetic valve leaflets. Thus, the structure of the pump attachment member is integrated with and completely compatible with the valve structure. Placing the pump a few centimeters away from the valve provides room for a high pressure balloon used to expand the stent.

### OBJECTS OF THE INVENTION

1. An object of the invention is to provide a miniature implantable blood pump suitable for long term use that can be implanted with minimally invasive surgery without the need for cardiopulmonary bypass.

2. An additional object of the invention is to provide a blood pump that can be implanted in the right or left ventricle and can be retained in place by an expandable stent placed in the pulmonary artery or aorta.

3. A further object of the invention is to provide a combined heart valve and blood pump that can be implanted in the same positions and manner described in object 1, and object 2 above.

4. It is an object of the invention to provide a blood pump that can be implanted across the aortic valve in the non-coronary cusp, of across the pulmonary artery valve, and retained there by an expandable stent placed in the aorta or pulmonary artery distal to the valve.

5. It is also an object of the invention to provide blood pumps that can be implanted in a ventricle by a trans-apical incision and then powered by a power cable exiting the heart via the apical incision.

6. It is another object of the invention to provide a less invasive heart assist device that can be used to treat elderly patients or patients who are too sick to tolerate a thoracotomy or sternoty procedure with cardiopulmonary bypass.

7. It is an object of the present invention to provide a minimally invasive heart assist device that will help patients achieve a rapid recovery and early discharge from the hospital, thus achieving overall cost savings.

### THE DRAWINGS

FIG. 1 is a drawing of a sectioned heart and aorta in which a device of the present invention, combining an expandable heart valve with a miniature intraventricular blood pump is shown drawn schematically.

FIG. 2 is a schematic drawing of a heart and aorta in which an expandable stent is shown in the aorta distal to the valve and a miniature blood pump is within the left ventricle, connected to the stent by three wires or sutures.

FIG. 3 is a similar drawing to FIG. 2 and shows a pump in the ventricle retained by the expandable stent by means of rod.

FIG. 4 is another schematic drawing of a heart and aorta, with an expandable stent in the aorta distal to the valve and an intraventricular pump attached to the stent by an outflow graft or cannula that passes across the aortic valve.

FIG. 5 is a schematic drawing of a miniature blood pump attached by an expandable stent affixed to the aorta distal to the valve. The pump is shown in a position such that it transverses the aortic valve.

FIG. 6 is a partially sectioned; partially schematic drawing where part of the aortic wall has been cut away permitting a view of the aortic valve leaflets from the aortic side. A miniature pump is shown in place anchored by an expandable fixation device.

FIG. 7 shows a pump and expandable fixation struts contained within a catheter used for surgical implantation.

FIG. 8 shows a miniature blood pump and fixation struts partially ejected from the catheter.

### SPECIFIC DESCRIPTION OF THE INVENTION

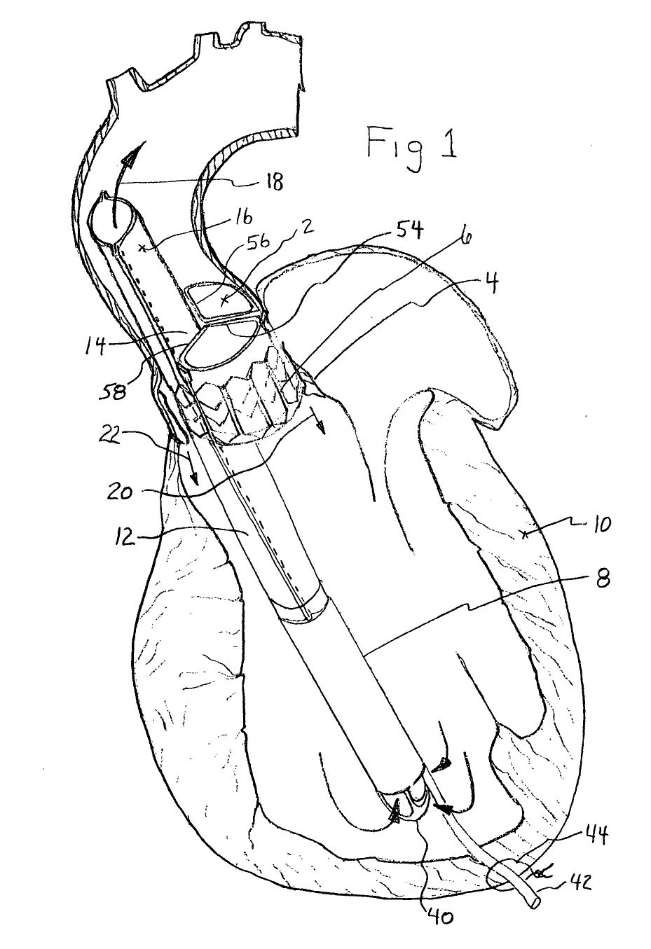

The present invention provides a miniature rotary blood pump located in left ventricle and anchored to the aorta or annulus of the aortic valve. Similar configurations to those shown in FIGS. 1-5 may be used in the right ventricle and pulmonary artery. The preferred embodiment, illustrated in FIG. 1, includes a prosthetic tissue valve 2, secured to the aorta at the annulus of the aortic valve 4, by an expandable metal stent 6. The term “expandable metal stent” is intended to include fenestrated metallic structures, metallic structures fabricated from wire or sheet stock, and hinged folding structures that can be inserted into a blood vessel when folded to a first smaller diameter, and then unfolded to a second larger diameter. A small axial flow blood pump 8, is positioned within the left ventricle 10. The outflow of the pump is connected to a conduit 12 within the ventricle that channels blood flow across the aortic valve at 14 in the position of the non-coronary cusp of the natural aortic valve. Since there is no coronary artery originating from the non-coronary sinus, the presence of the conduit does not block blood flow to a coronary artery as it could if the conduit passed through either other sinus of the aortic valve. A portion of the conduit 16 extends into the aorta distal to the valve and discharges blood as indicated by arrow 18. In reaction to the force of the blood streaming out of the pump and conduit, a force in the opposite direction is exerted on the anchoring stent, as shown by the arrows 20, 22. As a result of this force, the portion of the conduit 12.

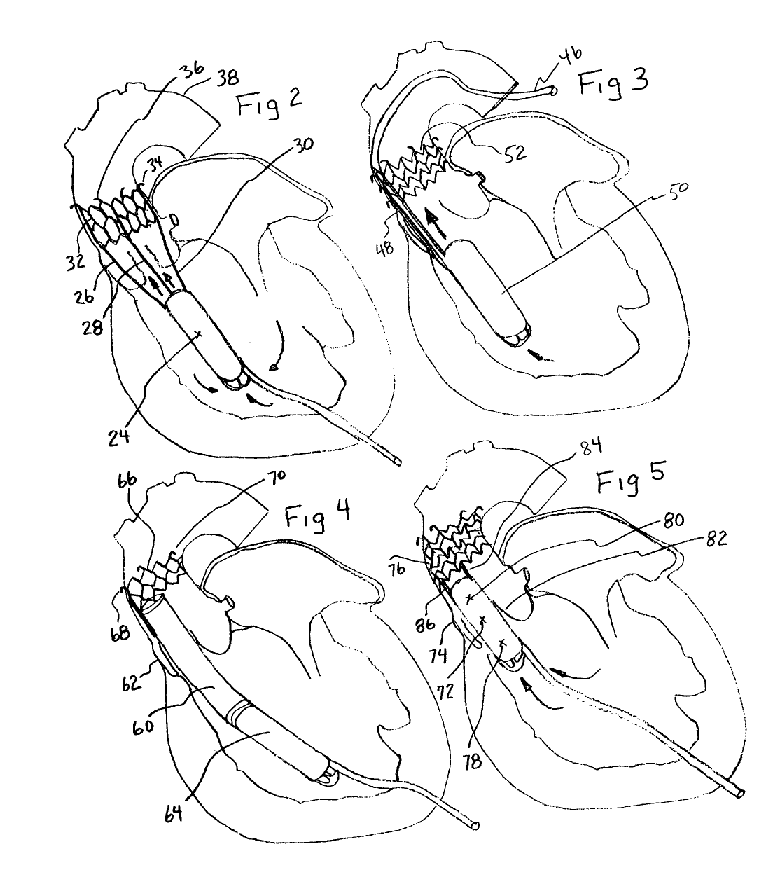

within the ventricle is in mechanical tension. Thus, the pump “hangs” from the stent by the flexible conduit. In another embodiment of the invention shown in FIG. 2 the blood pump 24 hangs by three filaments or cords 26, 28, 30 from a stent 32 affixed within the aorta 38. The filaments or cords are in tension as a result of the forces created by the pump and blood as it is thrust across the aortic valve. To better anchor the stent in light of this force, hooks 34, 36 that penetrate the aortic wall are provided on stent 32. These three cords may be located in the commisures of the natural valve where the leaflets join the aortic wall. At the commisures there is little motion of the leaflets so the cords can remain in place without eroding the leaflets.

Referring to FIG. 1, it is seen that the inflow side of the pump, where the flow enters as indicated by the arrows, may include a cage 40 to prevent it from becoming blocked with myocardial tissue. The pump power cable 42 transverses the apex of the ventricle where it is fixed and sealed by one or more sutures 44. The power cable will exit the ventricle at this position if the device has been introduced through an incision in the apex, as it can be. Other embodiments configured for insertion into the ventricle from the aortic root will have the power exiting through the aorta, as illustrated in FIG. 3, which shows the blood pump 50 anchored to the aorta by stent 52 by means of a hollow connecting rod 48 that passes the power wires 46 through it.

Again referring to the preferred embodiment of FIG. 1, the conduit tube 12 is preferentially fabricated of the same material as used for the prosthetic valve leaflets 54, 56. Thus, where conduit 12 contacts the valve leaflets, such as at 58, the material will be generally as resistant to erosion by mechanical rubbing of the leaflets against it as the leaflets are one against the other. The preferred material for the leaflets is treated pericardium, although other natural tissues, such as porcine valve leaflets and arteries, or dura mater, may be used. In some embodiments, synthetic materials such as polyurethane may be used. Additionally, the valve structure need not utilize just two leaflets as shown, but could be a monocusp valve.

FIG. 4 illustrates an embodiment where a blood conduit 60, placed across the non-coronary sinus of the natural aortic valve 62 connects a blood pump 64 to an expandable stent 66 anchored in the aorta. To increase the strength of the grip of the stent to the aortic wall, hooks 68, 70, may be included.

FIG. 5 shows an embodiment in which a very small blood pump, 72 is positioned across the non-coronary sinus of the aortic valve, 74, and is retained there by an expandable stent 76, located distal to it in the aorta, so that part of the pump, 78, is within the ventricle, and part of it, 80 is in the aorta. In this location the natural aortic valve leaflet 82 seals against the pump housing when the valve is closed. Flexible cords, 84, 86, or more rigid posts may be used to connect the blood pump to the stent.

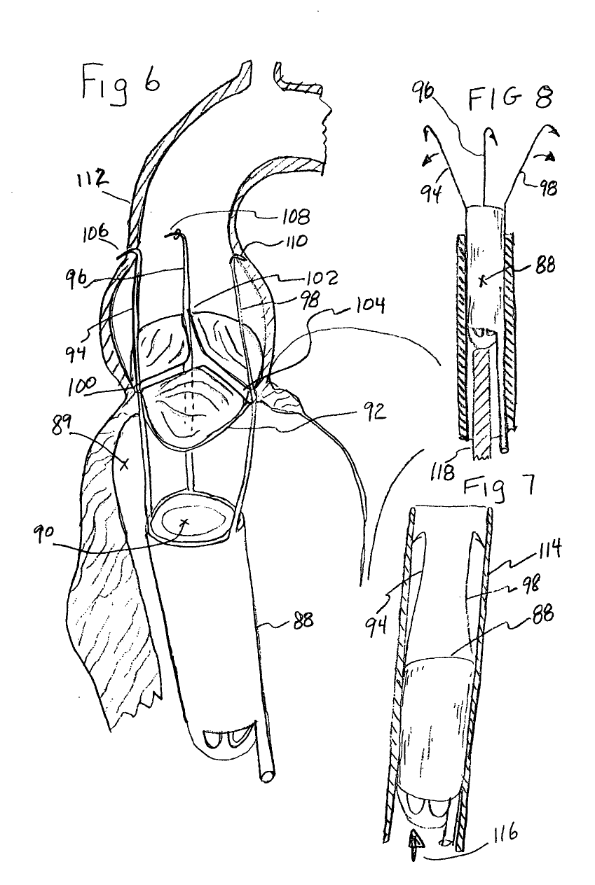

FIG. 6 shows an intraventricular blood pump 88, retained in the left ventricle 89 with its outflow 90 adjacent to the aortic valve 92. Three anchoring struts 94, 96, 98, preferentially made of a spring metal that is biocompatible, pass through the three commisures 100, 102, 104 of the aortic valve and are affixed to the pump. These struts are held into the aortic wall 112 by hooks 106, 108, 110, which may include barbs.

FIG. 7 shows the blood pump 88 within a catheter 114 used for surgical insertion of the pump and struts into their final position. Two spring struts 94, 98 are shown within the catheter 114. Arrow 116 indicates the direction that the pump and struts may be pushed to exit the catheter. In FIG. 8, a pusher rod, 118 is illustrated. During insertion, the catheter may be inserted across the aortic valve by a transapical approach and end of the catheter positioned in the aorta at the level where it is intended for the hooks to anchor the struts. Then, holding the pusher rod steady and pulling back on the catheter ejects the struts which spring outwardly and imbed the hooks and barbs into the aortic tissue. Further withdrawing the catheter and removing it and the pusher rod, leaves the pump implanted in the patient. A similar embodiment is envisioned having the power cable exit the pump in the direction of the outflow which permits this pump to be implanted from above the aortic valve, such as using a catheter system or thorascopic technique.

Additionally, rather than fixation using hooks or a stent, the three struts may have a small loop at the end, like the eye of a needle, through which fastening sutures may be placed, to suture the struts to the wall of the aorta. The ends of the struts may also include fabric coverings to permit suture attachment to the aortic or pulmonary artery wall.

In each of the embodiments illustrated, the preferred pump is a miniature axial flow pump utilizing thrombosis resistant blood immersed bearings and a hermetically sealed brushless DC motor to drive an impeller bearing rotor and thus pump the blood. Other suitable very small blood pumps including mixed flow pumps or tiny centrifugal pumps may be used.

The information disclosed in the description of the present invention is intended to be representative of the principles I have described. It will thus be seen that the objects of the invention set forth above and those made apparent from the preceding description are efficiently obtained and that certain changes may be made in the above articles and constructions without departing from the scope of the invention. It is intended that all matter contained in the above description and shown in the accompanying drawings shall be interpreted as illustrative but not in a limiting sense. It is also understood that the following claims are intended to cover all of the generic and specific features of the invention herein described and all statements of the scope of the invention which, as a matter of language, might be said to fall there between.

I claim:

1. A cardiac assist device including a miniature intraventricular blood pump structurally supported either entirely or partially within the right or left ventricle by one or more tension bearing posts, tubes, or filaments that are anchored within the aorta or pulmonary artery by connection to a fixation device placed therein.

2. The cardiac assist device of claim 1 in which the fixation device is an expandable stent.

3. The cardiac assist device of claim 1 in which the fixation device is an expandable stent deployed in the ascending aorta.

4. The cardiac assist device of claim 1 in which the fixation device is an expandable stent deployed in the annulus of the aortic valve.

5. The cardiac assist device of claim 1 in which the fixation device is a metallic structure including hooks that penetrate the aortic tissue to resist thrust forces created by the pump.

6. The cardiac assist device of claim 5 in which the fixation device is a metallic structure including three spring struts configured to be placed across the three commisures of the aortic or pulmonic valve.

7. The cardiac assist device of claim 1 in which said pump includes a power cable located on the pump end opposite the aortic valve, thus facilitating placement via the ventricular apex.

8. The cardiac assist device of claim 1 in which said pump includes a power cable located on the pump end closest to the aortic valve, thus facilitating placement via the ascending aorta.

9. A cardiac assist device including an intraventricular blood pump connected to a prosthetic tissue heart valve that is retained in the aorta by an expandable stent.

10. The cardiac assist device of claim 9 in which said valve is a pericardial tissue valve and the device includes a pump flow conduit tube formed of pericardium connected between the outflow of said pump and said expandable stent.

11. The cardiac assist device of claim 9 in which said expandable stent includes hooks or barbed hooks to help adsorb thrust forces generated by said pump.

12. The cardiac assist device of claim 9 in which said pump includes a power cable located on the pump end opposite the aortic valve, thus facilitating placement via the ventricular apex.

13. The cardiac assist device of claim 9 in which said pump includes a power cable located on the pump end closest to the aortic valve, thus facilitating placement via the ascending aorta.

14. The cardiac assist device of claim 9 in which said tissue valve is a porcine tissue valve or utilizes porcine valve leaflets.

15. The cardiac assist device of claim 9 in which said tissue valve includes three leaflets and said blood pump is retained entirely within the left ventricle in a position such the stream of blood exiting said pump is directed across said valve, from the left ventricle into the aorta or from the right ventricle into the pulmonary artery.

16. A cardiac assist device including an intraventricular blood pump affixed so that its outflow blood stream is directed across the aortic valve from within the left ventricular cavity into the aortic root without using a conduit that crosses the aortic valve, or from the right ventricle cavity into the pulmonary artery without using a conduit that crosses the pulmonic valve.

17. The cardiac assist device of claim 16 in which the pump is retained in place by an expandable stent.

18. The cardiac assist device of claim 16 in which the pump is retained in place by sutures.

19. The cardiac assist device of claim 16 in which the pump is retained in place by surgical staples or clips.

20. The cardiac assist device of claim 16 in which the fixation device is a metallic structure including three spring struts configured to be placed across the three commisures of the aortic or pulmonic valve, said spring struts including an eyelet or other structure configured to be affixed in place by sutures, staples, or clips, without using hooks.
